(19) United States
(12) Patent Application Publication
(10) Pub. No.: US 2019/0321607 A1
(43) Pub. Date: Oct. 24, 2019

(54) DEVICES AND METHODS FOR THE DILATION OF SINUS OSTIA AND OTHER BODY PASSAGES

(71) Applicants: Suresh S. Pai, Los Altos, CA (US); Celso Bagaoisan, Union City, CA (US)

(72) Inventors: Suresh S. Pai, Los Altos, CA (US); Celso Bagaoisan, Union City, CA (US)

(21) Appl. No.: 16/407,071

(22) Filed: May 8, 2019

Related U.S. Application Data
(63) Continuation of application No. PCT/US2017/060965, filed on May 9, 2017.
(60) Provisional application No. 62/419,965, filed on Nov. 9, 2016.

(51) Int. Cl. A61M 29/00; A61B 17/24; A61M 25/00

(52) U.S. Cl. CPC A61M 29/00; A61B 17/24; A61M 25/0074; A61M 2025/0004; A61M 25/0041

(57) ABSTRACT

Apparatus and methods are disclosed to dilate a sinus ostium or other body passage. In one embodiment, the apparatus includes an elongate inner member, one or more dilation members disposed concentrically around the inner member, and an outer sheath. The inner member includes a distal portion that extends beyond distal end of the dilation member(s). The sheath is disposed around the inner member and dilation member(s) and is at least partially expandable to accommodate advancement of the dilation member(s) relative to the inner member, e.g., to translate axial forces into radial dilating forces, and dilate surrounding tissue within which the distal portion is introduced. Optionally, an elastic cover is provided over at least a portion of the inner member distal portion to enhance constraining the sheath, e.g., after withdrawing the dilation member(s).

### RELATED APPLICATION DATA

The present application is a continuation of co-pending International Application No. PCT/US2017/060965, filed May 9, 2017, which claims benefit of U.S. provisional application Ser. No. 62/419,965, filed Nov. 9, 2016, the entire disclosures of which are expressly incorporated by reference.

### FIELD OF THE INVENTION

The present invention relates generally to devices and methods to dilate sinus ostia and other body passages.

### BACKGROUND

Chronic rhinosinusitis (CRS), or inflammation of the mucosal lining of the nose and paranasal sinuses, is a condition that reportedly affects 39 million people each year accounting for greater than 22 million office visits and 250,000 emergency room visits per year in the United States. Inflammation of the mucosal lining of the paranasal ostia restricts the natural drainage of mucous from the sinus cavity through mucocilliary clearance resulting in chronic infections within the sinus cavity. Symptoms of chronic rhinosinusitis include extreme pain, pressure, congestion, and difficulty breathing.

The first line of treatment for chronic rhinosinusitis is medical therapy including the administration of medications such as antihistamines, antibiotics, and anti-inflammatory agents such as steroids. Patients that are unresponsive or refractory to this medical therapy typically are considered for surgical intervention to help relieve the symptoms of the condition.

Functional endoscopic sinus surgery (“FESS”) is currently the most common type of surgery used to treat chronic sinusitis by remodeling the sinus anatomy via removal of mucosal tissue and bone. In a typical FESS procedure, an endoscope is inserted into the nose or nostril often along with a variety of other rigid, surgical instruments typically in a surgical operating room setting. These have traditionally included, but are not limited to the following tools: applicators, chisels, debriders, curettes, elevators, forceps, gouges, hooks, knives, saws, mallets, morselizers, needle holders, osteotomes, ostium seekers, probes, punches, backbiters, rasps, retractors, rongeurs, scissors, snares, specula, suction cannulae, and trocars. These instruments are then used to cut tissue and/or bone, cauterize, suction, debride, and the like in order to remodel paranasal sinuses and adjacent anatomy sufficiently to restore outflow of mucus. FESS, which was developed as an alternative to open surgical incisions and procedures, encompasses the use of an endoscope along with the listed tools to minimize patient trauma.

There is a school of thought that preservation of mucosal tissue during FESS procedures is valuable to long term clinical outcomes. In this regard, balloon dilatation of the sinuses has recently been introduced to the market by a number of companies as a minimally invasive approach to FESS. In this technique, the sinus surgeon places an endoscope and a guide catheter in the patient's sinus cavity usually via insertion through the nostrils.

Similar to cardiovascular applications, a surgeon advances a guide catheter with a preset geometry into a position that is close to the target sinus ostium after which a guidewire is introduced into the target sinus anatomy. A dilatation catheter is then loaded over the guidewire and advanced until the dilatation mechanism is in the sinus ostium, and the sinus ostia and outflow tract and ostium are expanded. In doing this sequence of steps, the bone structures underlying the sinus ostium that contact the dilatation catheter are remodeled and often fractured while preserving the overlying mucosa.

While an improvement over prior practice, these types of systems typically employ multiple working devices (e.g., an endoscope, sinus seeker, guide catheter, guidewire, dilatation catheter, and the like). The management and effective (often simultaneous) operation of these multiple tools in the surgical procedural setting can present a significant challenge to the surgeon. For example, at points in a procedure, the surgeon may be required to hold an endoscope in place in the sinus cavity while maintaining the position of a guide catheter and simultaneously advancing and directing a dilatation catheter into or through the target sinus ostium. Successful use of these often distinct, uncoupled devices requires intensive training and skill, and the requirement that many of these items be used concurrently may limit a physician's ability to provide the desired level of precision and accuracy.

The level of complexity of such procedures may be exacerbated when multiple sinus ostia are treated in the setting of a single procedure. In such cases, multiple guide catheters with varying tip angles or malleable formable tips and other apparatus are often required to successfully locate and cannulate the targeted sinus passageways. Due to patient to patient variation in sinus anatomy, the surgeon is required to stock each of these variations of the guide catheters in their disposable equipment inventories occupying valuable space in the operating room or healthcare facility and adding an economic burden to maintain these stock inventories for daily procedural use.

Recently, Entellus Medical (Minnesota, USA) introduced the XprESS Multi-Sinus Dilation Tool to address some of these shortcomings. The XprESS tool is a combination device including a ball-tipped malleable shaft with a through lumen that is intended to generally mimic the concept of the traditional sinus seeker used by surgeons. XprESS augments this sinus seeker-like component with a dilatation balloon catheter that is coaxially positioned over the outside wall of the malleable shaft.

The hub of the device allows the surgeon to apply a suction pressure to the distal tip of the malleable shaft, if desired, and the lumen of the malleable shaft may be used to position a guidewire if necessary. The hub also may include a Luer connector to allow attachment of a syringe to control inflation and deflation of a balloon. Finally, the hub may include a balloon slide mechanism that is intended to allow positioning of the balloon over the malleable shaft after it has been positioned at the desired sinus target. The malleable shaft is constructed from a material that allows it to be shaped by the surgeon in the field to a fixed geometry that the surgeon believes will be adequate to access the desired anatomy of the patient. While this product offers some differentiation with respect to the other dilatation tools in the market, it is aligned with the current commercially available products in the use of a high-pressure balloon to dilate the target sinus ostium.

The reliance on the infusion of fluid into an expandable member to achieve dilation of the ostium includes the inherent complexity of managing the syringes, fluid lines, and the like needed to inflate the expandable member. It becomes very challenging for a single operator to perform the sinus dilation procedure, as the surgeons hands are occupied with maintaining a clear view of the surgical field via an endoscope and maintaining a balloon dilation device in the proper position with respect to sinus ostium. An assisting nurse or other medical professional is needed to connect a syringe to the inflation line, inflate the balloon to the specified pressure, and deflate the balloon.

This process has a number of drawbacks. The method for controlling the pressure within the balloon is typically through the use of an indeflator, a controlled inflation device commonly used in cardiovascular t and peripheral vascular balloon angioplasty procedures, but not immediately familiar to medical personnel in the ear, nose, and throat field. The lack of familiarity is a potential source of errors (e.g., over or under-inflation) and may necessitate thorough training prior to use of the device. Furthermore, the use of high pressures near the orbit and skull base is risky in and of itself as a balloon rupture is by definition uncontrolled and can damage sensitive nerves and tissues the reside within the nasal cavity. Additionally, the requirement for an assisting nurse or other medical professional introduces a supplementary cost to dilation procedures that can be difficult for cost-sensitive community physicians to justify.

Thus, there exists a need for dilatation instruments and/or tools and methods that do not incorporate the high-pressure infusion of fluid as a method to dilate sinus ostia. These instruments should minimize trauma to the mucosa of the sinus ostia and surrounding tissues.

### SUMMARY

Described herein are devices and methods to dilate sinus ostia and/or other body passages within a patient's body. The devices generally include an elongate inner member that holds or carries one or more dilation members that may translate with respect to the elongate member, an outer sheath that overlies the inner member and one or more dilation members, an actuation member to control the position of the dilation members, and a handle.

In accordance with one embodiment, a dilation device comprises an elongate member that has distal and proximal ends. The elongate member may be sized to accommodate a range of sinus ostia and may be configured to have a constant outer diameter or variable or tapered outer diameter. The distal end of the elongate member may be formed into an atraumatic shape such as a hemisphere, sphere, and the like. The elongate member may be fabricated from metal and/or polymeric materials widely known in the art including, but not limited to, stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, composite materials and the like. The elongate member may optionally include at least one lumen extending between the proximal and distal ends.

In one embodiment, the elongate member may have a fixed or variable stiffness over its entire length or a portion of its length. For example, the elongate member may optionally comprise articulating joints or segments (e.g., accordion, transverse cuts, hinges, and the like) that allow the shape (e.g., the angle or curvature) of the elongate member to be adjusted. The articulating joints or segments may be fabricated from materials that are different from the bulk of the elongate member, and may be located at a single or varied position along the length of the elongate member.

For example, an articulating segment may allow the distal end of elongate member to bend from a relatively straight angle (i.e., generally 0°) to an angle of up to 120° from a longitudinal axis of the elongate member. Alternatively, the elongate member may be fabricated with a specified angle incorporated into the elongate member at a specified distance from the distal end of the elongate member.

For example, the distal tip may be angled at 0° to 20°, 20° to 40°, 40° to 60°, 60° to 80°, 80° to 100°, 100° to 120°, 120° to 140°, 140° to 160°, or 160° to 179° from the longitudinal axis of the main body of the elongate member. Exemplary degrees of angulation are 0°, 70° and 120°; however other angles may be specified to suit the particular target application. The elongate member or segments thereof may be malleable such that the curvature, arc, angle, or shape of the elongate member may be set and/or adjusted to a desired configuration in an analog manner prior to use and then maintained in said configuration.

Such a malleable, elongate member may comprise a single lumen or multiple lumens that may be plastically deformed to set the shape of the elongate member. Alternatively, the elongate member may include two or more lumens, one or more of which may contain a length of malleable rod or tube that may be plastically deformed to set the shape of the elongate member. Optionally, a second lumen may remain open to accept the insertion of other tools or device components, provide a method for infusion of fluid (e.g., gas, liquid) or other materials through the elongate member, or the like.

Alternatively, the lumen of a malleable tube may also provide a method for infusion of fluids or other materials. In one example, a lumen of the elongate member may be sized to accept a guidance device such as the PathAssist LED Light Fiber (Entellus Medical, Inc.), the Reliva Luma Sentry Sinus Illumination System (Acclarent), or other light emitting components, which may be used for transdermal illumination and the like. Alternatively, a lumen of the elongate member may be sized to accept emitters or receivers for use in optical or electromagnetic image guidance systems (e.g., the Fusion ENT Navigation System from Medtronic Xomed, the InstaTrak System from General Electric, Optical coherence tomography (OCT), and the like). In another example, the light emitting components, or emitters, or receivers, may be integrated into the body of a solid elongate member.

The at least one lumen may extend from the proximal end to the distal end of the elongate member. Each individual lumen may have a different shape in cross-section, including but not limited to elliptical (e.g., circular or oval), polygonal (e.g., convex, concave, regular, equilateral, equiangular, rectilinear, simple, combinations thereof, etc.), a combination of curved and straight segments, and the like. Alternatively, the at least one lumen or channel may terminate at a distance proximal to the distal end of the elongate member.

In the case of an elongate member including multiple lumens, each lumen may terminate at a uniform distance from the distal end of the elongate member or each lumen may terminate at a different distance from the distal end of the elongate member. Furthermore, the lumens may be spaced radially about the elongate member in any configuration. For example, a circular elongate member with four lumens may be designed such that the four lumens are located at approximately 0°, 90°, 180°, and 270° from the geometric center of the elongate member. In this example, the lumens or channels located at approximately 0° and 180° may terminate at the distal end of the elongate member while the lumens or channels located at 90° and 270° may terminate proximal to the distal end of the elongate member.

Optionally, each of the at least one lumens may further include at least one hole or opening extending from the interior of the lumen to the interior and/or exterior wall(s) of the elongate member. These hole(s) may be spaced at any position or in any pattern along the length of the lumen. For example, a series of three holes may be located in four lumens of the prior example spaced equidistantly from each other and the distal end of the elongate member over a total length of approximately four (4) centimeters. It should be clear to one of skill in the art that the specific size, location, number, and/or pattern of holes in each individual lumen, and the number, size, locations, and/or cross-sectional shape of the lumen(s) within the elongate member may be chosen to suite the particular application of a given embodiment of the device.

The elongate member may further include at least one flange or other external feature, such as protrusions, bumps, and the like, that increase the external dimension of the elongate member over a length of the elongate member. The at least one flange or other external feature may be an integrated part of the elongate member (e.g., formed through injection molding, machining, and/or another such technique known in the art) or, alternatively, an independent component that is temporarily or permanently joined or fixed to the elongate member using techniques known in the art including but not limited to one or more of bonding (e.g., adhesive bonding), welding, over-molding, threading/tapping, crimping, detents, and the like. In the case where a flange or other external feature is an independent part, the feature may be fabricated from metals and polymer widely known in the art including, but not limited to stainless steel, nickel, titanium, Nitinol, and alloys thereof, polyethylene, nylon, silicone, polyimide, acrylic, Pebax, polyurethane, PEEK, acetal, polycarbonate, polytetrafluoroethylene, combinations and copolymers thereof, and the like.

The at least one flange or other external feature may be located at any position along the length of the elongate member. For example, the at least one flange or other external feature may be joined at the distal tip of the elongate member, about zero to one centimeter (0.0-1.0 cm) proximal to the distal tip of the elongate member, about one to two centimeters (1.0-2.0 cm) proximal to the distal tip of the elongate member, about two to three centimeters (2.0-3.0 cm) proximal to the distal tip of the elongate member, and so on, e.g., in half (0.5) centimeter or less increments over the length of the elongate member.

In an exemplary embodiment, the elongate member may have a circular cross-section of a given diameter and include a flange of circular cross-section and a relatively larger diameter region located at the distal portion of the elongate member. Other shapes and/or orientations are contemplated, including but not limited to elliptical disks, wedges, polygonal or other geometric shapes, arms or bars, spirals, combinations thereof, and the like. Furthermore, any of these feature shapes may include a substantially constant or variable thickness and a linear, curved, or complex shape in transverse cross section. The use of multiple features as described herein located at different positions along the length of the elongate member is also contemplated.

In one embodiment, the dilation device further includes at least one dilation member that is slidably disposed about the elongate member. The dilation member may include a lumen sized to accept the elongate member. The lumen of the dilation member may be concentric with the outer diameter of the dilation member, or offset from the geometric center of the dilation member. The dilation member may be fabricated from metal and polymeric materials widely known in the art including, but not limited to, stainless steel, nickel, titanium, Nitinol, and alloys thereof, polyethylene, nylon, silicone, polyimide, acrylic, Pebax, polyurethane, PEEK, acetal, polycarbonate, polytetrafluoroethylene, combinations and copolymers thereof, composite materials and the like. The fabrication of the dilation member may include, but is not limited to ,the following processes: molding, extrusion, annealing, heat treating, winding, dipping, blow molding, machining, 3D printing, and the like.

The dilation member may have a constant outer profile or variable or tapered outer profile. In one example, the distal end of the dilation member is tapered such that there is a smooth transition between the outer surface of the elongate member and the outer surface of the dilation member. The taper may include a convex or concave curve, or combinations thereof. A linear or straight taper may have an angle with respect to the longitudinal axis of the dilation member that is from 0° to 10°, 10° to 20°, 20° to 30°, 30° to 40°, 40° to 50°, and so on, e.g., in 10° increments up to 90° (essentially no taper).

Optionally, the dilation member may possess a relatively low coefficient of friction. The low coefficient of friction may be obtained as an inherent characteristic of the component material of the dilation member (e.g., acetal/Delrin, fluoropolymers/Teflon, and the like), due to the application of a surface coating (e.g., PTFE, parylene, hydrophilic polymer coatings, and the like) to the internal or external surfaces of the dilation member, due to the application of a lubricant to the internal or external surfaces of the dilation member (e.g., silicone, graphite, molybdenum sulfate, and the like), or combinations thereof.

A low-friction inner surface may reduce the force needed to achieve translational motion of the dilation member with respect to the elongate member or other component disposed within a lumen of the dilation member. A low-friction outer surface reduces the force needed to achieve translational motion between the dilation member and the next outermost component of the dilation device. Optionally, the dilation member may have visual indicators to denote the relative position distal tip of dilation member with respect to either the elongate member or a local anatomical structure such as a sinus ostium under endoscopic evaluation.

The dilation member may further include at least one flange or other external or internal feature, such as a protrusion, bump, and the like, that increases the external dimension of the dilation member, or decreases the internal dimension of the dilation member over a length of the dilation member. The at least one flange or other external or internal feature may be an integrated part of the dilation member (e.g., formed through injection molding, machining, and/or another such technique known in the art) or, alternatively, an independent component that is temporarily or permanently joined or fixed to the dilation member using techniques known in the art including but not limited to bonding (e.g., adhesive bonding), welding, over-molding, threading/tapping, crimping, detents, combinations thereof, and the like.

In the case of a flange or other external or internal feature is an independent part, the feature may be fabricated from metal and/or polymer widely known in the art including, but not limited to stainless steel, nickel, titanium, Nitinol, and alloys thereof, polyethylene, nylon, silicone, polyimide, acrylic, Pebax, polyurethane, PEEK, acetal, polycarbonate, polytetrafluoroethylene, combinations and copolymers thereof, and the like. The at least one flange or other external or internal feature may be located at any position along the length of the dilation member. For example, the at least one flange or other external or internal feature may be joined at the distal tip of the elongate member, about zero to one centimeter (0.0-1.0 cm) proximal to the distal tip of the dilation member, about one to two centimeters (1.0-2.0 cm) proximal to the distal tip of the dilation member, about two to three centimeters (2.0-3.0 cm) proximal to the distal tip of the dilation member, and so on, e.g., in half (0.5) centimeter or less increments over the length of the dilation member.

In an exemplary embodiment, the dilation member may have a circular cross-section of a given diameter and include a flange of circular cross-section and a larger diameter located at the distal portion of the dilation member. In another exemplary embodiment, a dilation member including a lumen of a given diameter may further include an extruded post that extends into the lumen of the dilation member. Other shapes and/or orientations are contemplated, including, but not limited to, elliptical disks, wedges, polygonal or other geometric shapes, arms or bars, spirals, combinations thereof, and the like. Furthermore, any of these feature shapes may comprise a constant or variable thickness and a linear, curved, or complex shape in transverse cross section. The use of multiple features as described herein located at different positions along the length of the dilation member is also contemplated.

In addition to the aforementioned extruded or protruding features, the dilation member may include one or more channels, cavities, detents, and/or other shapes or features that are carved out of the dilation member. In the cases of dilation members that include a lumen or lumens, channels, cavities, detents, or other shapes or features may extend from the external surface of the dilation member to one of the lumens to form a port or pathway between an internal space of the dilation member and the space external to the dilation member. Alternatively, the channels, cavities, detents, or other shapes or features may not fully penetrate the wall of the dilation member.

It is contemplated that a given dilation member may include any combination of extruded features on the internal and/or external surfaces of the dilation member, in addition to any combination of channels, cavities, ports, detents, and the like, on the internal and/or external surfaces of the dilation member. In one example, the dilation member may be an extruded length of tubing with a tapered distal end, a lumen, and several internal features extending into the lumen. In another example, the dilation member may be a coil that is wound to have a profile that begins at a low outer diameter at a proximal section, tapers up to a second, larger outer diameter at a point distal to the proximal section, maintains the larger outer diameter for a given length, then tapers down to the initial outer diameter at the distal-most portion. The size of the wire or extrusion used to create the coil will dictate the flexibility and handling characteristics of the dilation member as well as the size and shape of the lumen. It should be clear to one of skill in the art that a wide range of mechanical characteristics (e.g., linear flexibility, radial strength, turning radius, compressive strength, and the like) and/or geometric profiles may be obtained by varying the material components and means of fabrication of the dilation member.

In an exemplary embodiment, the dilation device may have two or more dilation members. In one example of an embodiment including multiple dilation members, a second dilation member may be slidably disposed about the first dilation member. The second dilation member may include a lumen that is sized to accept the first dilation member. The lumen of the second dilation member may be concentric with the outer diameter of the second dilation member, or offset from the geometric center of the second dilation member.

The second dilation member may possess all the of the characteristics (e.g., construction material, stiffness, geometry, coefficient of friction, visual indicators, extruded features, cavities, channels, ports, and the like), as previously described for the first dilation member. The second dilation member may have a distal taper that is chosen to transition smoothly to the distal portion of the first dilation member when the first and second dilation members are fully translated in the distal direction with respect to the elongate member. In this manner, a smooth profile is produced that runs from the second dilation member, to the first dilation member, and finally to the distal end of the elongate member.

It should be clear to one of skill in the art that additional dilation members may be arranged in a similar manner to that of the first and second dilation members. In one such an embodiment, a third dilation member would relate to the second dilation member as the second dilation member relates to the first dilation member. Furthermore, it is contemplated that multiple dilation members may be components of the device described herein, and these dilation members may have a myriad of geometric arrangements with respect to each other and the elongate member. For example, a subset of the dilation members may be concentrically oriented about the elongate member while a second subset of dilation members may be offset from the concentric axis of the elongate member. While these examples serve to illustrate the concept of multiple dilation members, it should be clear to one of skill in the art that any number or arrangement of dilation members is contemplated.

In accordance with one embodiment, a sheath may be arranged around an outermost dilation member of the one or more dilation members. The sheath may be a thin liner that is elastically deformable or a folded membrane that is not elastically deformable or combination thereof. The sheath may be bonded to the elongate member, to a handle of the device, or to an intermediate component that is neither the elongate member nor the handle of the device using methods known to the art including, but not limited to bonding, welding, ultrasonic welding, over-molding, threading/tapping, crimping, press or interference fits, combinations thereof, and the like.

The sheath may be fabricated from materials including, but not limited to aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, natural rubber, nitrile rubber, silicone rubber, combinations and copolymers thereof, and the like. The inner and outer surfaces of the sheath are preferably low-friction. The low coefficient of friction may be obtained as an inherent characteristic of the component material of the sheath (e.g., acetal/Delrin, fluoropolymers/Teflon, etc.), due to the application of a surface coating (e.g., PTFE, parylene, hydrophilic polymer coatings, and the like) to the internal or external surfaces of the sheath, due to the application of a lubricant to the internal or external surfaces of the sheath (e.g., silicone, graphite, molybdenum sulfate, etc.), or combinations thereof.

A low-friction inner surface may reduce the force needed to achieve translational motion of the outermost dilation member with respect to the sheath. For example, a low-friction outer surface allows the device to easily pass over anatomical structures such as nasal mucosa without applying excessive shear forces to the anatomical structures. This is beneficial in that lower shear forces will mitigate damage to sensitive tissue that may be caused by the passage of the one or more dilation members. For example, sequential passage of dilation members, in the absence of a sheath such as those described in U.S. Pat. No. 8,777,926, Chang et. al. (Acclarent, Inc.) may be traumatic to the surrounding mucosa in contrast to the apparatus and methods herein, which may protect the mucosa during sequential dilation via the sheath. The sheath may have visual indicators to denote the position of the device of the invention with respect to the target anatomy (e.g., a sinus ostium) under endoscopic evaluation. The sheath may be folded and/or wrapped about the elongate member and/or the outermost dilation member prior to use.

In accordance with another embodiment, the device may also optionally include an elastic cover that is arranged about the sheath and extends along a portion of the length of the elongate member, e.g., from a distal tip of the elongate member along a region of the distal portion. The optional elastic cover may help maintain and/or provide a low dimensional profile and/or a groomed surface at the distal end of the device, e.g., after multiple dilations. The low profile groomed surface may also facilitate crossing sinus ostia and other tight spaces in the nasal cavity or other body lumens. A single dilation device is typically used for dilation of multiple sinus ostial targets and as such a low profile groomed surface is desirable.

The elastic cover may extend from the distal end of the elongate member in the proximal direction, e.g., from about zero to one centimeter (0.0-1.0 cm) proximal to the distal tip of the elongate member, about one to two centimeters (1.0-2.0 cm) proximal to the distal tip of the elongate member, about two to three centimeters (2.0-3.0 cm) proximal to the distal tip of the elongate member, and so on, e.g., in half (0.5) centimeter or less increments over the length of the elongate member.

The elastic cover may be fabricated from materials known in the art including, but not limited to aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, natural rubber, nitrile rubber, silicone rubber, thermoplastic elastomers (e.g., C-Flex®), combinations and copolymers thereof, and the like. The elastic cover may be bonded to the distal portion of the sheath using known methods and/or materials including, but not limited to bonding, solvent bonding, welding, ultrasonic welding, over-molding, threading/tapping, crimping, press or interference fits, combinations thereof, and the like.

Optionally, the elastic cover may be bonded to the sheath at other points along the length of the elastic cover. Optionally, the elastic cover may have a low-friction or lubricious outer surface, and/or may be able to expand and contract without wrinkling, folding, or pancaking as other components of the device (e.g., one or more dilation members) pass beneath the elastic cover.

The device may further include a handle fabricated from metal and/or polymeric materials widely known in the art including, but not limited to, stainless steel, nickel, titanium, and alloys thereof, polyethylene, nylon, silicone, polyimide, acrylic, Pebax, polyurethane, PEEK, acetal, polycarbonate, polytetrafluoroethylene, combinations and copolymers thereof, composite materials and the like. Differing embodiments of the handle may interact with the dilation member or members in a variety of ways. In one example, the handle may include an actuator (e.g., a push button, lever, knob, gear, thumb slider switch or the like as known in the art) that is connected, either directly or through intermediate components, to a dilation member and, e.g., that enables the surgeon to advance, or as desired, to retract the dilation member about the elongate member.

Optionally, the handle may further include one or more grooves, cavities, channels, and the like, e.g., as needed to accommodate the action of a specific actuator or actuators. For example, a handle that includes a slider-style actuator may also include an open channel that enables the slider-style actuator to connect to a dilator member whose proximal portion resides within the handle. Likewise, the handle may include one or more ports that are sized to allow portions of the elongate member and any number of dilation members to reside within the handle.

In another example, the handle may include multiple actuators that are joined, either directly or through intermediate components, to multiple dilator members. In yet another example, the handle may include a single actuator that is joined to one of several dilator members wherein the dilator members are slidably connected (either reversibly or irreversibly) to one another. In this manner, advancement of the actuator may translate more than one dilator member about the elongate member.

Furthermore, the cadence and/or sequence of translation of the various dilator members may be controlled and/or defined by the arrangement of internal and/or external features on each of the dilator members. The proximal end of the elongate member may be joined to the handle, either directly or through an intermediate component. The actuator may be a simple machine that incorporates mechanical advantage such as a ratcheting mechanism, or a rack and pinion mechanism to convert radial motion into linear motion. Alternatively, the actuator may have some degree of automation, as exemplified in the use of a spring and release mechanism to drive the distal translation of the dilator member(s) at the push of a button.

Optionally, the actuator may also be fully motorized, in that the user may control the distal and proximal translation of the dilation member(s) via manipulation of a motor that is linked to one or more of the dilation members. The handle may further include a stop, detent, and/or other feature that limits the distance that the dilation member(s) may travel relative to the elongate member. The handle, or portions thereof, may be in fluid communication with one or more lumens of the dilation member(s) and/or the elongate member, if those components in turn comprise one or more lumens.

Optionally, the outer surface of the handle may be contoured to facilitate ergonomic considerations for the physician operator, or to promote ideal means of holding and/or handling the device of the invention. The handle may optionally include one or more visual indicators and/or instructions to the user, and/or may further include markers, e.g., to enable a surgeon to determine the position of the device, or a component of the device, with respect to the anatomy of the patient. These markers may include visual indicators such as colored bands, radiologic indicators such as radio-opaque metals, alloys, and other materials, emitters or receivers for use in optical or electromagnetic image guidance systems (e.g., the Fusion ENT Navigation System from Medtronic Xomed, the InstaTrak System from General Electric, and the like), light emitting components for transdermal illumination, and the like.

The handle and actuators may be configured to allow for single use distal translation of the dilation member(s), or alternatively, may be configured to enable cyclic proximal to distal to proximal translation of the dilation member(s). In the latter case, the device may also include components that enable the sheath to be re-folded to a low profile after the initial translation cycle.

In accordance with one embodiment, the device may include a handle, an elongate member, a dilation member, a sheath, and an optional elastic cover. The proximal portion of the elongate member is enclosed within the handle, and the proximal end of the elongate member is joined to the handle. The distal portion of the elongate member comprises an approximately 120° curve and the elongate member does not include a lumen.

In exemplary embodiments, the dilation member may be fabricated from stainless steel wire and may be arranged slidably and coaxially with the elongate member. The distal portion of the dilation member is a linear taper that is smallest at the distal tip of the dilation member and reaches its maximum diameter at a point proximal to the distal end of the dilation member. The outer diameter of the largest section of the dilation member is sized to be sufficiently large to dilate the target sinus ostium. This outer diameter may be about 2-3 millimeters in size, about 3-4 millimeters in size, about 4-5 millimeters in size, about 5-6 millimeters in size, or larger.

The proximal end of the dilation member is joined to the distal end of an intermediate component; the intermediate component is coaxially and slidably arranged with the elongate member and positioned proximal to the dilation member along the elongate member. The handle may be sized and/or contoured to fit comfortably in one hand, and further may include a channel that runs substantially parallel to the long axis of the handle as well as a distal port.

In one embodiment, the distal port is a cylindrical column that extends from the distal face of the handle and has a core lumen that is sized to slidably accept the elongate member and intermediate member. The elongate member and the intermediate member pass through the distal port of the handle. The proximal portion of the intermediate member further comprises an actuator that extends from the intermediate member and through the channel in the handle to form a slide. A proximal section of the sheath is bonded to the outer surface of the distal port of the handle. The sheath extends from the distal port of the handle to the distal end of the elongate member and is sized to fit over the dilation member. The sheath may be folded to obtain a low cross-sectional profile.

The optional elastic cover is sized to fit over the sheath, and the distal end of the elastic cover is bonded to the distal end of the sheath. The device may be used to dilate a maxillary sinus ostium, e.g., by inserting the proximal end of the device into the nasal cavity of a patient and advancing the distal end of the elongate member across the target maxillary sinus ostium under endoscopic visualization. The approximately 120° curve at the distal end of the elongate member facilitates placement of the elongate member around the uncinate process.

When desired, the physician advances the actuator towards the distal end of the handle. This movement in turn advances the intermediate member and dilation member in the distal direction. The presence of the sheath and optional elastic cover shields the sensitive nasal mucosa from the shear forces created by the distal motion of the dilation member, allowing the nasal mucosa to be subjected to a purely radial pressure from the dilation member as the dilation member advances linearly (i.e., linear radial dilation). As the taper of the dilation member passes through the ostium, the pressure against the walls of the ostium break the wafer-like bone and dilate the ostium to the largest diameter of the taper on the distal end of the dilation member. Once dilated, the physician retracts the actuator and draws the dilation member in the proximal direction to remove it from the ostium. The optional elastic cover contains and collapses the sheath as the dilation member translates in the proximal dimension and the progressively smaller diameters of the taper are presented to the ostium. The physician may then remove the device from the patient. Alternatively, but sub-optimally, the device may be removed without retracting the dilation member, if the physician desires.

While this is an exemplary embodiment of the devices and methods for use herein, it should be clear to one of skill in the art that the general principles described may be extended to devices that encompass all of the designs disclosed herein. For example, a device that includes electromagnetic receivers and emitters may be used in concert with an electromagnetic guidance system to replace or supplement endoscopic visualization of the procedure. Alternatively, a device that includes an elongate member with a lumen sized to accept a light-emitting device and a corresponding proximal access port in the handle may use the technique of transdermal illumination to confirm placement of the distal end of the device across the target sinus ostium. This may be accomplished by passing the light-emitting device through the access port in the handle, down the lumen of the elongate member, and out of the tip of the elongate member. In yet another embodiment, the apparatus may include an elongate member with a lumen sized to accept an Optical coherence tomography imaging device that provides optical guidance to the physician regarding the tissues and anatomy surrounding the tip and/or body of the elongate member thereby aiding location of the target ostium.

In another example, the device may include one or more of several dilation members that are stacked in a telescopic fashion to provide a method for controlling the dilation of the target sinus ostium via the successively larger diameters of the dilation members. In this example, each dilation member may be color coded to correspond to a specific outer diameter (e.g., blue=4 mm, green=5 mm, purple=6 mm, etc.). Another embodiment may include an elongate member that is malleable; and the operator may be able to reshape the elongate member of this embodiment to be compatible with multiple sinus ostia (e.g., a 120° bend for the maxillary sinus ostium, a 70° bend for the frontal sinus ostium, etc.).

Other aspects and features including the need for and use of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A shows a transverse view of a distal portion of an exemplary embodiment of an apparatus for dilating sinus ostia.

FIG. 1B shows a transverse view of a proximal portion of the apparatus shown FIG. 1A.

FIG. 2A-2C are transverse views illustrating a mechanism of action of an embodiment of an apparatus including two dilation members that may be included in the apparatus shown in FIGS. 1A and 1B. A sheath and elastic cover are not shown for clarity.

FIG. 3A shows a transverse view of the distal portion of another embodiment of an apparatus including a coiled wire dilation member.

FIG. 3B shows a transverse view of the proximal portion of the apparatus shown in FIG. 3A.

FIG. 4 is a flowchart demonstrating several exemplary methods for using an apparatus, such as those shown in FIGS. 1A-3B to dilate a target sinus ostium.

### DETAILED DESCRIPTION OF THE EXEMPLARY EMBODIMENTS

Before the exemplary embodiments are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range, is also specifically disclosed. Each smaller range between any stated value or intervening value in a stated range and any other stated or intervening value in that stated range is encompassed within the devices and methods herein. The upper and lower limits of these smaller ranges may independently be included or excluded in the range, and each range where either, neither or both limits are included in the smaller ranges is also encompassed within the devices and methods, subject to any specifically excluded limit in the stated range. Where the stated range includes one or both of the limits, ranges excluding either or both of those included limits are also included in the devices and methods herein.

Unless defined otherwise, all technical and scientific terms used herein have the same meaning as commonly understood by one of ordinary skill in the art to which the devices and methods herein belong. Although any methods and materials similar or equivalent to those described herein may be used in the practice or testing of the present invention, some potential and/or exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymer and equivalents thereof known to those skilled in the art, and so forth.

The publications discussed herein are provided solely for their disclosure prior to the filing date of the present application. Nothing herein is to be construed as an admission that the devices and methods herein are not entitled to antedate such publication by virtue of prior invention. Further, the dates of publication provided may be different from the actual publication dates which may need to be independently confirmed.

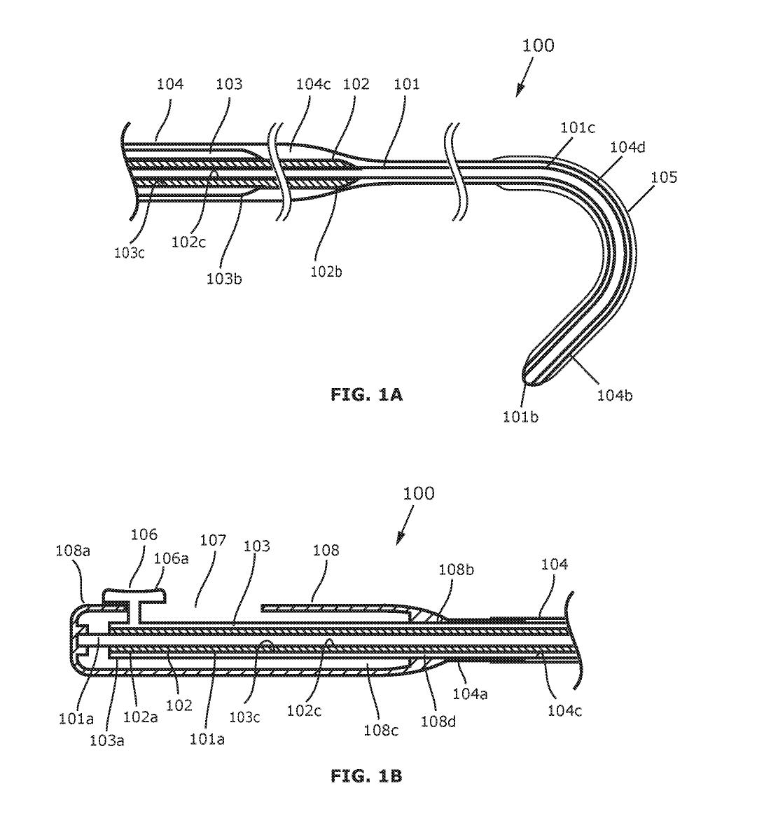

Turning to the drawings, FIGS. 1A and 1B show an exemplary embodiment of a catheter or apparatus 100 for dilating sinus ostia. Generally, the apparatus 100 includes an elongate inner member 101, a first dilation member 102, and second dilation member 103, a sheath 104, and an optional elastic cover or cuff 105. Optionally, the apparatus 100 may be used in cooperation with one or more additional devices, such as an endoscope, guidewire, light guidance system, Optical coherence tomography imaging systems, electromagnetic navigation systems, and the like (not shown), to provide a system for dilating a sinus ostium, as described elsewhere herein.

The inner member 101 generally includes a proximal end 101a, a distal end 101b sized for introduction into a patient's body, and a distal portion 101c extend a predetermined length from the distal end 101b. In one embodiment, the inner member 101 may be a solid rod or wire, although alternatively, the inner member 101 may be hollow, optionally including a lumen (not shown) extending between the proximal and distal ends 101a, 101b. For example, in an alternative embodiment, the inner member 101 may include an instrument lumen for receiving a guidewire or other guide element (not shown).

The first and second dilation members 102, 103 also include proximal ends 102a, 103a, distal ends 102b, 103b, and also include a lumen 102c, 103c extending between the proximal and distal ends. For example, the inner member 101 may be disposed within the lumen 102c such that the first dilation member 102 is slidably disposed about the elongate member 101, and the first dilation member 102 may be disposed within lumen 103c such that the second dilation member 103 is slidably disposed about the first dilation member 102.

The sheath 104 also includes a proximal end 104a and a distal end 104b, and includes a lumen 104c that receives the other components of the apparatus 100. In particular, the sheath 104 surrounds a length of the inner member 101, e.g., from the distal end 101b proximally to a handle 108 that encloses the proximal ends 101a, 102a, 103a of the members 101, 102, 103.

At least a distal portion 104d of the sheath 104 is configured to expand and/or unfold, e.g., to accommodate the relatively large diameter of dilation member 103 during advancement, as described elsewhere herein. In this example, the inner member 101, first dilation member 102, and second dilation member 103 are substantially circular in cross section and are coaxially aligned with respect to each other.

As shown in FIG. 1A, the distal portion 101c of the inner member 101 is shown in a curved or bent shape, e.g., defining an arc of extend at least approximately 120°. For example, the distal portion 101c may be biased to a curved or other curvilinear shape, yet may be resiliently straightened or otherwise manipulated, e.g., to facilitate advancement through tortuous anatomy. Alternatively, the distal portion 101c may be biased to a straight or other shape and/or otherwise sufficiently flexible, yet have sufficient column strength to allow advancement by manipulating the handle 108. The optional elastic cover 105 is positioned over the distal portions of elongate member 101 and sheath 104, and is bonded or otherwise attached to the distal end of sheath 104. For example, the elastic cover 105 may provide a cuff adjacent the distal end 101b of the inner member 101, which may enhance collapse of the sheath 104, e.g., if the apparatus 100 is used to multiple dilations and/or introduced sequentially into multiple passages. The distal ends 102b, 103b of the first dilation member 102 and the second dilation member 103 are concentrically tapered, e.g., terminating in rounded, conical, or otherwise atraumatic distal tips, to provide a smooth, continuous transition when both dilation members are fully translated in the distal direction.

FIG. 1B details a proximal portion of the apparatus 100 showing the handle 108 and proximal ends 101a, 102a, 103a, therein. The proximal end 104a of the sheath 104 is permanently attached to a distal end 108b of the handle 108, e.g., by one or more of bonding, fusing sonic welding, interference fit, and the like, while the inner member 101, the first dilator member 102, and the second dilator member 103 pass through a distal port 108d in the distal end 108b and into an interior 108 of handle 108. The proximal end 101a of the inner member 101 is permanently attached to a proximal end 108a of handle 108, e.g., by one or more of cooperating connectors, interference fit, bonding, fusing, sonic welding, and the like.

The handle 108 further includes a channel 107, in a side wall of the handle housing, that provides communication between the external environment and the interior 108c of handle 108 and/or provide a track for receiving an actuator for manipulating the components of the apparatus 100 in a desired manner. For example, as shown in FIG. 1B, the second dilation member 103 may include an actuator 106 that extends from a proximal section of the second dilation member 103, through the channel 107, and terminates in a pad or button 106a that may be used to translate the second dilation member 103 proximally or distally with respect to the handle 108 and the inner member 101 during use, as described further elsewhere herein.

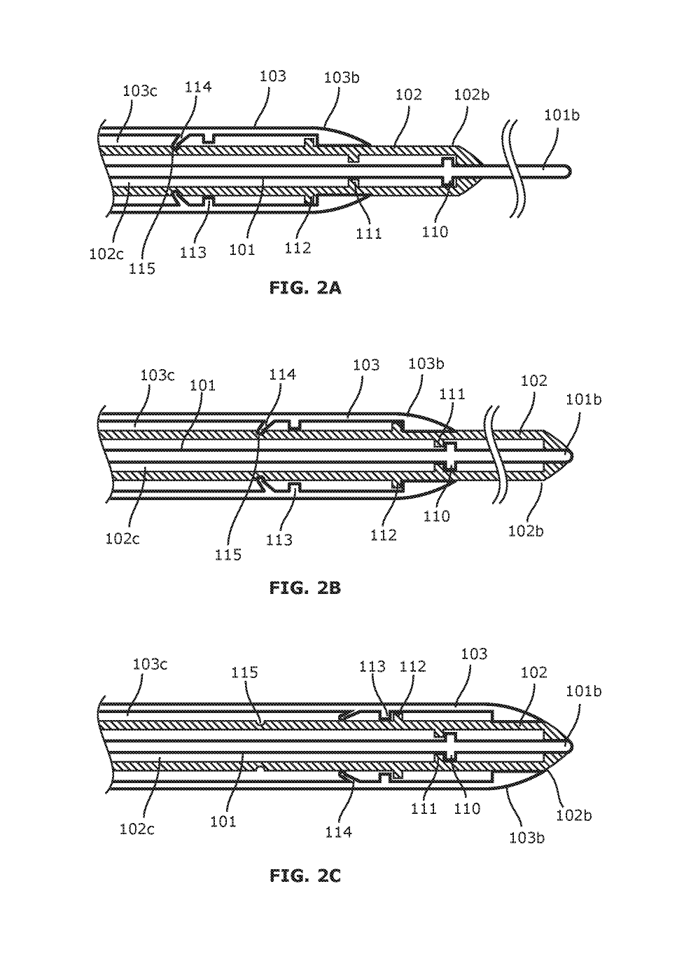

FIGS. 2A-2C show details of internal component of the apparatus 100 in FIGS. 1A and 1B. The sheath 104 and elastic cover 105 are omitted in FIGS. 2A-2C for clarity. FIGS. 2A-2C demonstrate an exemplary mechanism for advancing, in sequence, the first dilation member 102 and the second dilation member 103 with a single distal motion of the actuator 106 shown in FIG. 1B.

In particular, FIGS. 2A-2C are cross-sections of a distal portion of the apparatus 100, showing the relative location of the inner member 101, the first dilation member 102 and the second dilation member 103 during actuation, e.g., from an initial or proximal position, shown in FIG. 2A, to a first distal position, shown in FIG. 2B, to a second or final distal position, shown in FIG. 2C. As described further below, the inner member 101 and first and second dilation members 102, 103 include one or more cooperating elements that limit relative motion, e.g., to allow initial and simultaneous advancement of the first and second dilation members 102, 103 from the initial positon to the first distal position, and then further advancement of the second dilation member 103 alone to the second distal positon.

For example, in the example shown, the inner member 101 includes a flange, tab, detent, or other element 110 that extends outwardly at an intermediate location, e.g., proximal to the distal portion (not shown). The first dilation member 102 also includes a plurality of flanges, tabs, detents, or other elements, namely an internal flange 111, an external flange 112, and a detent 115. The internal flange 111 is located at a first location within the first dilation member lumen 102c proximal to the distal end 102b of the first dilation member 102 and is sized to interfere with the flange 110 of the inner member 101. The external flange 112 is located at a second location proximal to the internal flange 111 and extends outwardly within the second dilation member lumen 103c, and the detent 115 is located at a third location proximal to the external flange 112. The second dilation member 103 also includes a plurality of flanges, tabs, detents, or other elements, namely a second internal flange 113 at a first location and a living hinge 114 located at a second location proximal to second internal flange 113, both within the second dilation member lumen 103c. It will be appreciated that the axial distance between the elements on the first and second dilation members 102, 103 may be set to limit axial movement of the first and second dilation members 102, 103 relative to each other and the inner member 101.

FIG. 2A shows the apparatus 100 in the initial position corresponding to the actuator 106 shown in FIG. 1B in the proximal-most position in the channel 107 (also shown in FIG. 1B). In the initial position, the living hinge 114 of the second dilation member 103 is engaged with the detent 115 of the first dilation member 102. This reversibly joins the first dilation member 102 to the second dilation member 103. For example, until the translational force of the actuator 106 acting on the second dilation member 103 in the distal direction is greater than the engagement force of the living hinge 114 and detent 115, the first dilation member 102 and second dilation member 103 will move as a single unit.

FIG. 2B shows the apparatus 100 after the first and second dilation members 102, 103 are simultaneously advanced to the first position corresponding to the actuator 106 shown in FIG. 1B being between the proximal and distal ends of channel 107 (also shown in FIG. 1B). As shown, the relative lengths of the inner member 101 and first and second dilation members 102, 103 may be set such that, in the first distal position, the distal end 102b of the first dilation member 101 has reached the distal 101b end of the inner member 101. At the first distal position, the internal flange 111 has contacted the flange 110, preventing further distal motion of the first dilation member 102.

Consequently, further force applied to the actuator 106 in the distal direction will act to separate or disengage the living hinge 114 and detent 115. Once the living hinge 114 and detent 115 separate, the second dilation member 103 will translate in the distal direction with respect to the first dilation member 102 and the inner member 101 to the second distal position. FIG. 2C shows the apparatus 100 in the second distal position corresponding to the actuator 106 shown in FIG. 1B being at the distal end of the channel 107 (also shown in FIG. 1B), and the distal end 103b of the second dilation member 103 has reached the distal end of the first dilation member 102, e.g., such that the tapered tips are disposed concentrically to provide a substantially continuous tapered tip for the apparatus 100. In the second distal position, the living hinge 114 and detent 115 have separated and the second internal flange 113 has contacted the external flange 112, thereby preventing further distal translation of the second dilation member 103.

During use, the distal portion 101c of the inner member 101 may be introduced into a target sinus ostium or other body passage (not shown), with the first and second dilation members in the initial position shown in FIG. 2A. Optionally, the distal portion 101c may be introduced through a guide sheath, a lumen of an endoscope, or other device into the target ostium (not shown). Once positioned at a desired location, the actuator 106 may be advanced to direct the first and second dilation members 102, 103 to the first distal position, shown in FIG. 2B, and then direct the second dilation member 103 to the second distal position, shown in FIG. 2C in one continuous motion.

During this action, the first and second dilation members 102, 103 advance into and through the sheath 104 and elastic cover (not shown). The sheath 104 and/or elastic cover 105 may provide a path within which the first and second dilation members 102, 103 advance, e.g., to reduce resistance to advancement and/or minimize tearing or other damage to adjacent tissue. For example, the sheath 104 and/or elastic cover 105 may be formed from lubricious material and/or may include an internal and/or external coating.

In addition, the sheath 104 and/or elastic cover 105 may expand to accommodate passage of the dilation members 102, 103. For example, in one embodiment, the sheath 104 and/or elastic cover 105 may be formed from elastic material such that they may elastically expand as the dilation members 102, 103 are advanced. Alternatively, the sheath 104 and/or elastic cover 105 may be formed from inelastic material that is folded, twisted, or otherwise constrained around the distal portion 101c of the inner member 101. For example, the material may be bonded to the inner member or itself using a low bond adhesive or the material may be sufficiently tacky to remain in its constrained configuration. In this alternative, advancement of the dilation members 102, 103 causes the material to unfold or otherwise open to accommodate the dilation members 102, 103 passing through. Thus, in this alternative, the sheath 103 and/or elastic cover 105 may have an inner diameter corresponding to the outer diameter of the second dilation member 103.

In addition, the elastic cover 105 may provide a cuff adjacent the distal end 101b of the inner member 101, which may enhance collapse of the sheath 104, e.g., if the apparatus 100 is used to multiple dilations and/or introduced sequentially into multiple passages, as described further elsewhere herein.

With the second dilation member 103 at the second distal position shown in FIG. 2C, the target sinus ostium would be dilated to the diameter of the second dilation member 103 (plus the thickness of the sheath and elastic cover, not shown). While these figures show one embodiment for enabling a telescopic translation of two dilation members with a single actuator, it should be clear to one of skill in the art that any number of additional dilation members may be included in the apparatus 100. In a similar manner, the living hinge and detent mechanism of reversibly joining two dilation members is only one mechanism of many that may be employed for this purpose. For example, a keyed track on the internal surface of the handle interacting with corresponding features on the outermost dilation member would serve a similar purpose.

Optionally, the components may be free to move proximally from the second distal position, e.g., if desired to retract the second dilation member 103 and/or first dilation member 102 after being advanced. For example, the actuator 106 could be moved between its proximal and distal positions multiple time to advance and retract the dilation members 102, 103 multiple times, if desired. Alternatively, in the second distal position, the first and second dilation members 102, 103 may include cooperating locking elements that engage to prevent subsequent withdrawal of the second dilation member 103. For example, the first dilation member 102 could include a second detent (not shown) that receives the living hinge 114 at the position shown in FIG. 2C. In addition or alternatively, the first dilation member 102 and inner member 101 may include cooperating locking elements that engage to prevent subsequent proximal movement of the first dilation member 102 once advanced to the first distal position. In a further alternative, a lock may be provided that interacts with the actuator 106 and/or handle 108.

Optionally, the apparatus 100 may be used to perform multiple dilations. For example, after withdrawing the dilation members 102, 103 back to the initial position, the elastic cover 105 may enhance collapse of the sheath 104, which may facilitate removal of the apparatus 100 from the nasal ostium (or other body passage) and introduction into another nasal ostium (or other body passage), whereupon the dilation members 102, 103 may be advanced again to dilate another target location.

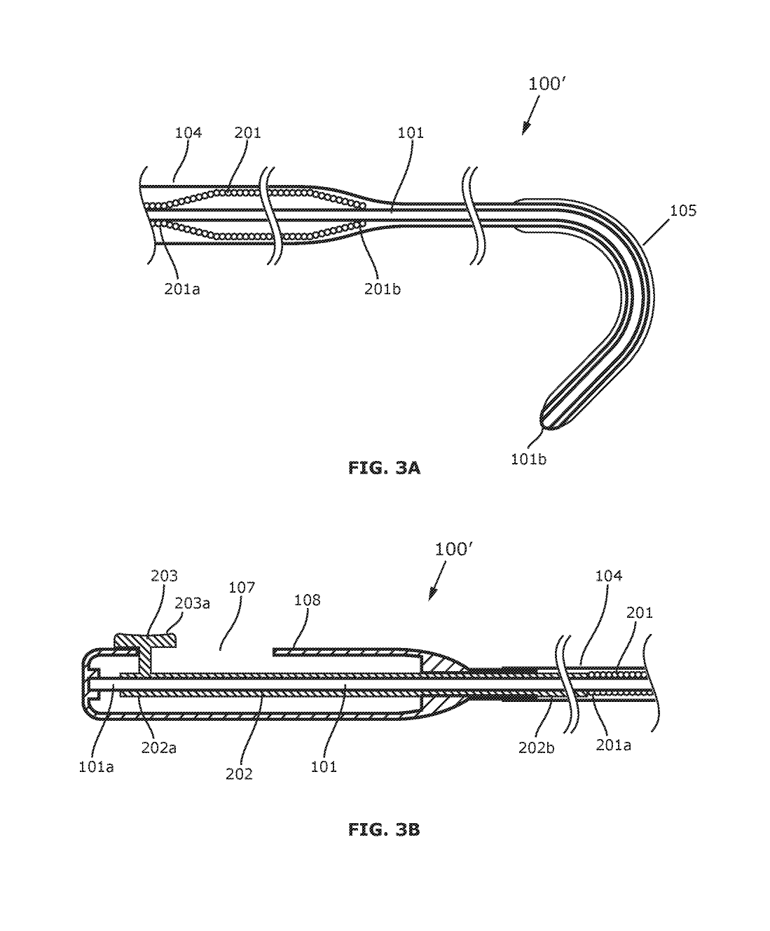

Turning to FIGS. 3A and 3B, another exemplary embodiment of an apparatus 100′ is shown, generally similar to the apparatus 100, showing cross-sectional details of distal and proximal portions of the apparatus 100′. The apparatus 100′ includes an elongate inner member 101, but replaces the first and second dilation members 102, 103 with a single dilation member 201 in the shape of a tapered spring or coil. The coil member 201 may include a cylindrical main region between proximal and distal tapered regions, with adjacent loops of the coil substantially contacting one another in the initial position. In this embodiment, a proximal end 201 of the dilation member 201 is joined to a distal end 202b of an intermediate member 202. The intermediate member 202 is coupled to and/or includes an actuator 203 that extends from a proximal end 202a of the intermediate member 202, through channel 107 in handle 108, and terminates in a pad or button 203a that can be used to simultaneously translate the intermediate member 202 and dilation member 201 proximally or distally with respect to the handle 108 and the inner member 101 generally similar to the previous embodiment. As the coil dilation member 201 is advanced, the coils may facilitate advancement over the distal portion 101c of the inner member 101, e.g., if positioned within a non-linear passage.

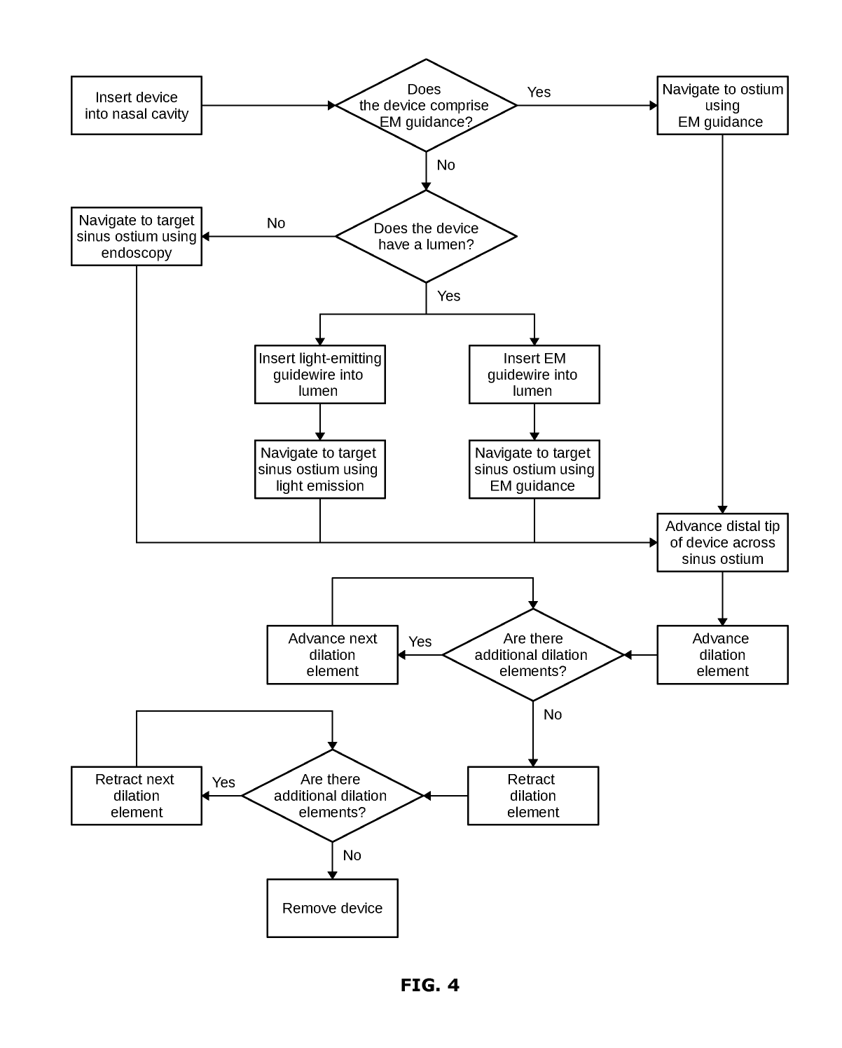

FIG. 4 is a flowchart showing exemplary methods of using a device, such as any of the apparatus described herein, to dilate a sinus ostium (not shown). The initial step in any of the methods of use described herein is to insert a distal portion of the device into the nasal cavity. If the device does not include any receivers or emitters for use in an electromagnetic guidance system, and the device does not include a lumen that is able to accept accessory devices, the physician user may navigate the device to the target sinus ostium using endoscopic visualization. The distal tip of the device is then advanced across the target sinus ostium and the first dilation element is translated distally to dilate the ostium. If the device comprises additional dilation elements, these are advanced in turn until all the dilation elements have been passed into the ostium. Alternatively (not shown), if less than the full complement of dilation elements have been engaged and the sinus ostium has been dilated to an acceptable dimension, the physician user may move on to the step of retracting the dilation elements in the proximal direction. This may be done for a single dilation element, or in succession for a series of dilation elements. Once all of the dilation elements have been retracted, the device may be removed from the patient.

If the device of the invention comprises receivers or emitters for use in an electromagnetic guidance system, the physician user may use electromagnetic guidance in addition to endoscopic visualization to navigate to the target sinus ostium. At this point, the remainder of the method of use is the same as previously described. If the device does not have receivers or emitters for use in an electromagnetic guidance system, but does possess a lumen that is accessible to accessory devices, a light-emitting guidewire may be inserted through the lumen of the device and used to navigate to the target sinus ostium using a combination of transdermal light emission and endoscopic visualization. Alternatively, a guidewire possessing receivers or emitters for use in an electromagnetic guidance system may be inserted through the lumen of the device and used to navigate to the target sinus ostium using a combination of electromagnetic guidance and endoscopic visualization. In both cases, the remainder of the method of use will be consistent with the prior description once the distal tip of the device has been advanced across the sinus ostium.

The foregoing disclosure of the exemplary embodiments has been presented for purposes of illustration and description. It is not intended to be exhaustive or to limit the invention to the precise forms disclosed. Many variations and modifications of the embodiments described herein will be apparent to one of ordinary skill in the art in light of the above disclosure. For example, although the embodiments have been described as particularly useful for dilating sinus ostia, the apparatus and methods described herein may also be used for dilating other body passages within a patient's body, e.g., a blood vessel within a patient's vasculature, a passage within the urinary tract, a passage within the gastrointestinal system, e.g., within the rectum, intestines, esophagus, and the like.

Further, in describing representative embodiments, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for dilating a body passage, comprising:
an elongate inner member comprising a proximal end, a distal end, and a distal portion;
a first dilation member comprising a proximal end, a distal end, and a first lumen extending between the first dilation member proximal and distal ends, the inner member disposed within the first lumen such that the distal portion extends beyond the first dilation member distal end;
a second dilation member comprising a proximal end, a distal end, and a second lumen extending between the second dilation member proximal and distal ends, the first dilation member disposed within the second lumen such that the first dilation member distal end is disposed distally to the second dilation member distal end;
a sheath disposed concentrically around at least the distal portion of the inner member and at least around the distal ends of the first and second dilation members, the sheath being expandable to accommodate advancement of the second dilation member over the distal portion;
a handle coupled to a proximal end of the sheath and including an interior housing the proximal ends of the inner member, first dilation member, and second dilation member, the handle comprising an actuator coupled to the second dilation member for advancing the first and second dilation members relative to the inner member.

2. The apparatus of claim 1, wherein the first and second dilation members comprise cooperating elements that interact such that, when the actuator is manipulated to advance the second dilation member, the first dilation member advances simultaneously with the second dilation member.

3. The apparatus of claim 2, wherein the cooperating elements are configured such that, when the first and second dilation members are advanced to a first distal position, the detents disengage, whereupon further manipulation of the actuator causes the second dilation member to advance to a second distal position while the first dilation member stops advancing.

4. The apparatus of claim 3, wherein the first and second dilation members comprise cooperating stops that limit further advancement of the second dilation member beyond the second distal position.

5. The apparatus of claim 2, wherein the first dilation member and the inner member comprise cooperating stops that engage when the first dilation member is advanced to the first distal position to prevent further distal movement of the first dilation member.

6. The apparatus of claim 3, wherein, in the second distal position, the distal ends of the first and second dilation members are disposed concentrically around one another.

7. The apparatus of claim 6, wherein the first dilation member terminates at a first tapered distal tip and the second dilation member terminates at a second tapered distal tip, and wherein, in the second distal position, the first and second tapered distal tips are aligned to provide a substantially continuous tapered tip in the second distal position.

8. The apparatus of claim 7, wherein the actuator is configured to advance the first and second dilation members such that the first and second tapered distal tips are aligned with a distal tip of the inner member in the second distal position.

9. The apparatus of claim 1, wherein the distal portion of the inner member is biased to a curvilinear shape.

10. The apparatus of claim 1, wherein the sheath is formed from elastic material such that the sheath elastically expands around the distal portion during advancement of the second dilation member.

11. The apparatus of claim 1, wherein the sheath is formed from inelastic material that is rolled, folded, or otherwise constrained around the distal portion, the sheath configured to unroll, unfold, or otherwise open to accommodate advancement of the second dilation member.

12. The apparatus of claim 1, further comprising an elastic cover disposed over a region of the distal portion of the inner member.

13. The apparatus of claim 1, wherein the elastic cover is biased inwardly to enhance collapse of the sheath within the elastic cover after removal of the dilation members.

14. An apparatus for dilating a body passage, comprising:
an elongate inner member comprising a proximal end, a distal end, and a distal portion;
a dilation member comprising a proximal end, a distal end, and a lumen extending between the dilation member proximal and distal ends, the inner member disposed within the lumen such that the distal portion extends beyond the dilation member distal end;
a sheath disposed concentrically around at least the distal portion of the inner member and at least around the distal end of the dilation member, the sheath being expandable to accommodate advancement of the dilation member over the distal portion; and
a handle coupled to a proximal end of the sheath and including an interior housing the proximal ends of the inner member and dilation member, the handle comprising an actuator coupled to the dilation member for advancing the dilation member relative to the inner member within the sheath.

15. The apparatus of claim 14, wherein the sheath is configured to reduce friction during advancement of the dilation member.

16. The apparatus of claim 14, wherein the sheath is configured to translate axial forces during advancement of the dilation member into radial forces to dilate surrounding tissue.

17. The apparatus of claim 14, wherein the sheath is configured to minimize shear applied to surrounding tissue during advancement of the dilation member.

18. The apparatus of any claim 15, wherein the dilation member comprises a coil member including a cylindrical main region between proximal and distal tapered regions, with adjacent loops of the coil substantially contacting one another.

19-27. (canceled)

28. A method for dilating a sinus ostium, comprising:
providing an apparatus comprising an inner member including a distal portion and first and second dilation members disposed on the inner member in an initial position proximal to the distal portion, and an sheath overlying the distal portion;
introducing the distal portion of the inner member into a nasal cavity until positioned within a target ostium;
manipulating an actuator of the apparatus to simultaneously advance the first and second dilation members over the distal portion through the sheath to direct a distal end of the first dilation member into the target ostium to dilate the target ostium; and
further manipulating the actuator to advance the second dilation member further to direct a distal end of the second dilation member to further dilate the target ostium.

29. The method of claim 28, wherein the sheath translates axial forces during advancement of the first and second dilation members into radial forces to dilate surrounding tissue while minimizing shear applied to the surrounding tissue.

30-48. (canceled)
